(19) United States
(12) Patent Application Publication
(10) Pub. No.: US 2019/0230931 A1
(43) Pub. Date: Aug. 1, 2019

(54) SURFACE TREATMENTS

(71) Applicants: Nicolai Vladimirovich BOVIN, Moscow (RU); Stephen Micheal HENRY, Auckland (NZ)

(72) Inventors: Nicolai Vladimirovich BOVIN, Moscow (RU); Stephen Micheal HENRY, Auckland (NZ); Igor Leonidovich RODIONOV, Pushchino (RU)

(21) Appl. No.: 16/294,757

(22) Filed: Mar. 6, 2019

Related U.S. Application Data
(63) Continuation of application No. 15/585,296, filed on May 3, 2017, now abandoned.
Application No. 15/585,296 is a continuation-in-part of application No. PCT/IB2016/052735, filed on May 12, 2016.
Application No. PCT/IB2016/052735 is a continuation-in-part of application No. PCT/NZ2015/050181, filed on Nov. 3, 2015.

(30) Foreign Application Priority Data
Nov. 3, 2014 (AU) 2014904423
May 20, 2015 (AU) 2015901844

(51) Int. Cl. A01N 57/12; A61L 15/46; A61L 31/16; A61L 31/14; A61L 31/02; A61L 27/54; A61L 27/04; A61L 27/50

(52) U.S. Cl. CPC A01N 57/12; A61L 15/46; A61L 31/16; A61L 31/14; A61L 2300/404; A61L 27/54; A61L 27/042; A61L 27/50; A61L 2300/22; A61L 31/022

(57) ABSTRACT

An antimicrobial surface treatment method comprising the step of contacting a stainless-steel surface with an aqueous dispersion of at least one functional-lipid construct where the lipid moiety is a phosphatidylethanolamine and the functional moiety confers the antimicrobial activity.

This application is a continuation of U.S. Ser. No. 15/585,296 filed May 3, 2017, which is a continuation-in-part of PCT/IB2016/052735 filed May 12, 2016, which claims priority to PCT/NZ2015/050181 filed Nov. 3, 2015 and AU 2015901844 filed May 20, 2015. U.S. Ser. No. 15/585,296 is also a continuation-in-part of PCT/NZ2015/050181, which claims priority to AU 2015901844 filed May 20, 2015, and AU 2014904423 filed Nov. 3, 2014, the contents of each of which are hereby incorporated by reference.

### TECHNICAL FIELD

The invention relates to methods of treating surfaces to provide advantageous properties, e.g. antimicrobial properties, and lipid constructs for use in such methods. In particular, the invention relates to methods of treating surfaces using selenide-lipid and polycation-lipid, in particular polyamine-lipid constructs.

### BACKGROUND ART

As stated in the publication of Gallo et al (2014) it is expected that the projected increased usage of implantable devices in medicine will result in a natural rise in the number of infections related to these cases. The current knowledge of antimicrobial surface treatments suitable for prevention of infection is reviewed. Surface treatment modalities include minimizing bacterial adhesion, biofilm formation inhibition and bactericidal action.

The publications of Reid and others disclose biocidal formulations including a selenium (Se) compound. The selenium compounds may be deposited on a surface and covalently or non-covalently associated with it. A broad range of selenium compounds are proposed, including compounds of the formula RSeX where R is an aliphatic or phenolic group and X is a protecting group.

Cationic lipids have primarily been developed for use in liposomal gene delivery as an alternative to viral-based gene delivery, but have also been identified as having bactericidal activity. Common cationic lipid classes include N-[1-(2,3-dioleyloxy)propyl]-N,N,N-trimethylammonium chloride (DOTMA) and 3β[N-(N′,N′-dimethylaminoethane)-carbamoyl] cholesterol (DC-Chol). At least partly because of the low efficiency of lipofection the vast majority of clinical trials of gene therapy have used alternative means of gene delivery. The further development of cationic lipids has sought to improve the efficiency of lipofection.

The publications of Behr et al (1989) and Remy et al (1994) disclose spermine-lipid conjugates where the lipid is a phosphatidylethanolamine (DOPES and DPPES). Conjugation is via the carboxyl function of a functionalised L-5-carboxyspermine derivative. The conjugates are used in the preparation of compacted lipopolyamine-coated plasmids. The coated plasmids are used in a transfection procedure.

The publication of Byk et al (1989) discloses structure-activity relationships amongst a series of cationic-lipids developed for use in DNA transfer. Amongst the lipoamines evaluated in these studies, the polyamine geometry was shown to have an influence on transfection efficiency.

The publication of Randazzo et al (2009) discloses an exploration of the dual functionality of cationic lipids in the context of gene transfer and bactericidal activity. The cationic lipids demonstrated to possess these activities comprised a sterol moiety as the lipid component.

The publication of Kato et al (2003) discloses a method of adhering otherwise non-adherent cells to surfaces using a biocompatible anchor.

It is an object of the present invention to provide a method of treating the surface of surgical dressings and implants using lipid constructs that is effective to reduce the incidence of postoperative infections. It is an object of the present invention to provide a method of treating a surface that is effective to promote the adherence of micro-dimensioned particles to the surface. It is an object of the present invention to provide selenium and polycation-lipid constructs for use in these methods. These objects are to be read in the alternative with the object at least to provide a useful choice in the selection of such methods or constructs.

### DISCLOSURE OF INVENTION

In a first aspect the invention provides an antimicrobial surface treatment method comprising the step of contacting the surface of an object with an aqueous dispersion of at least one functional-lipid construct where the lipid is a di-acyl, di-alkenyl or di-alkyl glycerophospholipid and the functional moiety of the construct confers the antimicrobial activity.

Preferably, the object is a surgical dressing or implant. More preferably, the object is a surgical implant. Most preferably, the surface is stainless steel.

Preferably, the aqueous dispersion is devoid of detergents and organic solvents. More preferably, the aqueous dispersion consists essentially of water and the at least one functional-lipid construct.

Preferably, the lipid is a diacylglycerophospholipid. Preferably, the lipid is a diacylglycerophospholipid where the acyl substituents are aliphatic C₁₄₋₂₀ acyl substituents. More preferably, the lipid is a diacylglycerophospholipid where the acyl substituents are aliphatic C₁₄₋₂₀ acyl substituents independently selected from the group consisting of: myristoyl (C₁₄), palmitoyl (C₁₆), stearoyl (C₁₈) and oleoyl (C₁₈). Preferably, the diacylglycerophospholipid is phosphatidylethanolamine. Most preferably, the diacylglycerophospholipid is dioleoyl phosphatidylethanolamine (DOPE).

Preferably, the functional moiety is selected from the group consisting of: selenide and polycation. More preferably, the functional moiety is selected from the group consisting of: cyanoselenide and polyamine. Most preferably, the functional moiety is cyanoselenide or spermine.

Preferably, the antimicrobial surface treatment is an antibacterial surface treatment. More preferably, the antimicrobial surface treatment is a bactericidal surface treatment.

Preferably, the contacting the surface is by immersing the object in the dispersion for a time sufficient to provide the antimicrobial surface treatment. More preferably, the time is less than 60 seconds. Yet more preferably, the time is less than 30 seconds. Most preferably, the time is less than 10 seconds.

Preferably, the dispersion is sonicated whilst the object is immersed.

Preferably, the concentration of the construct in the dispersion is sufficient to provide the antimicrobial surface treatment. More preferably, the concentration is less than 1 mg/mL of construct.

In a first embodiment of the first aspect the invention provides an antimicrobial surface treatment method comprising the step of contacting the surface with an aqueous dispersion of a selenide-lipid construct where the lipid is a di-acyl, di-alkenyl or di-alkyl glycerophospholipid. Preferably, the lipid is a diacylglycerolipid. More preferably, the lipid is a diacyl-glycerophospholipid. Most preferably, the lipid is phosphatidylethanolamine.

In a second embodiment of the first aspect the invention provides a method of treating the surface of a surgical implant comprising the step of contacting the surface with an aqueous dispersion of a polycation-lipid construct where the polycation is an N¹-acylated polyamine and the lipid is a di-acyl, di-alkenyl or di-alkyl glycerophospholipid. Preferably, the lipid is a diacyl-glycerophospholipid. Most preferably, the lipid is phosphatidylethanolamine.

In the second embodiment of the first aspect of the invention, the polycation-lipid construct is preferably of the structure:

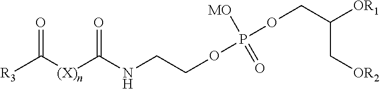

where M is a monovalent cation, n is the integer 3, 4 or 5 and X is the divalent radical methylene (—CH₂—), R₁ and R₂ are independently selected from the group consisting of C₁₄₋₂₀ saturated, mono- or di- unsaturated, unbranched acyl groups, and R₃ is the N¹-acylated polyamine.

In a second aspect the invention provides a selenide-lipid construct of the structure:

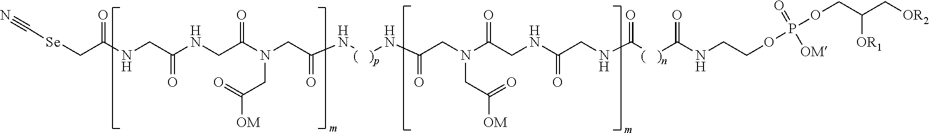

where:
m is the integer 1,2,3 or 4; preferably the integer 1, 2 or 4; most preferably the integer 2;
n is the integer 3, 4 or 5; most preferably the integer 4;
p is the integer 1, 2 or 3; most preferably the integer 2;
q is the integer 1, 2 or 3; most preferably the integer 1;
M is a monovalent substituent; preferably the monovalent substituent CH₃ or H; most preferably the monovalent substituent H;
M′ is a monovalent cation or substituent; preferably the monovalent cation H⁺, K⁺ or Na⁺; most preferably the monovalent cation H⁺; and
R₁ and R₂ are independently an aliphatic C₁₄₋₂₀ acyl, aliphatic C₁₄₋₂₀ alkenyl or aliphatic C₁₄₋₂₀ alkyl substituent; preferably a substituent selected from the group consisting of: myristyl, palmityl, stearyl, arachidyl, palmitoleoyl, petroselenyl, oleoyl, elaidyl, vaccenyl and gondoyl; most preferably the aliphatic C₁₈ alkenyl substituent oleoyl.

In a third aspect the invention provides a polycation-lipid construct of the structure:

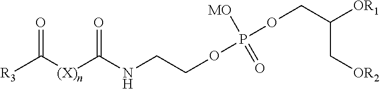

where X is —CH₂— and n is the integer 3, 4 or 5; R₁ and R₂ are independently selected from the group consisting of aliphatic C₁₄₋₂₀ acyl, aliphatic C₁₄₋₂₀ alkenyl or aliphatic C₁₄₋₂₀ alkyl; and R₃ is an N¹-acylated polyamine.

Preferably, R₁ and R₂ are independently selected from the group consisting of: C₁₄₋₂₀ acyl groups that are unbranched and saturated or mono-unsaturated. More preferably, R₁ and R₂ are independently selected from the group consisting of: myristyl, palmityl, stearyl, arachidyl, palmitoleoyl, petroselenyl, oleoyl, elaidyl, vaccenyl and gondoyl. Most preferably, R₁ and R₂ are the aliphatic C₁₈ alkenyl substituent oleoyl.

Preferably, R₃ is of the structure:

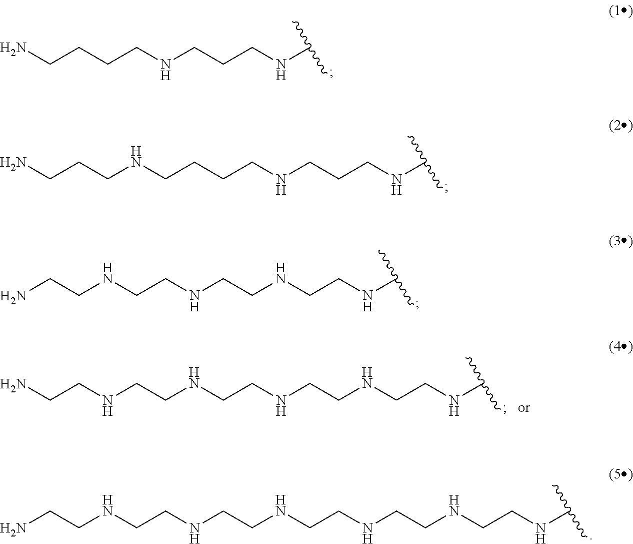 [SMILES: *NCCCNCCCCN ; *NCCCNCCCCNCCCN ; *NCCNCCNCCNCCN ; *NCCNCCNCCNCCNCCN ; *NCCNCCNCCNCCNCCNCCN]

In a fourth aspect the invention provides a surface treatment preparation consisting essentially of a dispersion in water of at least one construct of the second or third aspects of the invention.

In the description and claims of this specification the following acronyms, terms and phrases have the meaning provided: “alicyclic” means cyclic aliphatic; “aliphatic” means alkanes, alkenes or alkynes or their derivatives and is used as a descriptor for compounds that do not have the special stability of aromatics; “alkanes” means a saturated hydrocarbon of the general formula CₙH₂ₙ₊₂; “alkenes” means unsaturated hydrocarbons that contain one or more double carbon-carbon bonds; “alkynes” means unsaturated hydrocarbons that contain one or more triple carbon-carbon bonds; “aromatic” means containing a benzene ring or having similar chemical properties; “Boc” means tert-butoxycarbonyl; “Boc₃Spm” means (N¹,N⁴,N⁹-tri-tert-butoxycarbonyl)-1,12-diamino-4,9-diazadodecane; “comprising” means “including”, “containing” or “characterized by” and does not exclude any additional element, ingredient or step; “consisting essentially of” means excluding any element, ingredient or step that is a material limitation; “consisting of” means excluding any element, ingredient or step not specified except for impurities and other incidentals; “dispersible in water” means dispersible in pure, deionised water at 25° C. in the absence of organic solvents or surfactants to provide a dispersion at a concentration of at least 1 μmol/mL and “water dispersible” has a corresponding meaning; “DOPE” means 1,2-O-dioleoyl-sn-glycero-3-phosphatidylethanolamine; “DSPE” means 1,2-O-distereoyl-sn-glycero-3-phosphatidylethanolamine; “hydrophilic” means having a tendency to mix with, dissolve in, or be wetted by water and “hydrophilicity” has a corresponding meaning; “hydrophobic” means having a tendency to repel or fail to mix with water and “hydrophobicity” has a corresponding meaning; “monovalent cation” means an ion having a single positive charge and includes the monovalent cations H⁺, Na⁺, K⁺ or (CH₃CH₂)₃N⁺; “N¹-acylation” means the attachment of an acyl group (RCO—) at a terminal, primary amine of the longest chain of the molecule and “N¹-acylated” has a corresponding meaning; “polyamine” means an unbranched organic compound comprising three or more amine functions including at least two primary amino (—NH₂) functions; and “Spm” (or “spm”) means spermine.

The terms “first”, “second”, “third”, etc. used with reference to elements, features or integers of the matter defined in the Statement of Invention and Claims, or when used with reference to alternative embodiments of the invention are not intended to imply an order of preference.

Where concentrations or ratios of reagents or solvents are specified the concentration or ratio specified is the initial concentration or ratio of the reagents or solvents. Where values are expressed to one or more decimal places standard rounding applies. For example, 1.7 encompasses the range 1.650 recurring to 1.749 recurring.

In the absence of further limitation, the use of plain bonds in the representations of the structures of compounds encompasses (where applicable) the diastereomers, enantiomers and mixtures thereof of the compounds. In the representations of the structures or substructures of compounds the repeat of a divalent radical is represented by:

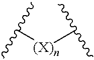

where —X— is the divalent radical repeated n times. Where the divalent radical is methylene (—CH₂—) the repeat of this divalent radical is represented by:

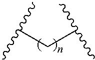

To facilitate the description of the preparation and use of the constructs the following designations are used:

“-Ad-” designates the substructure:

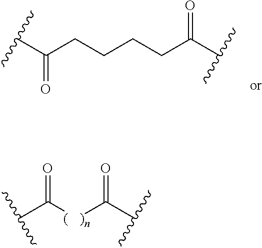

where n is the integer 4;

“-CMG(m)-” designates the substructure:

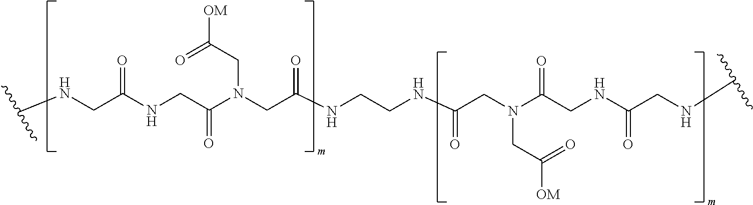

where m is the integer 1, 2, 3 or 4 and M is a monovalent substituent; and “-DOPE” designates the substituent of the structure:

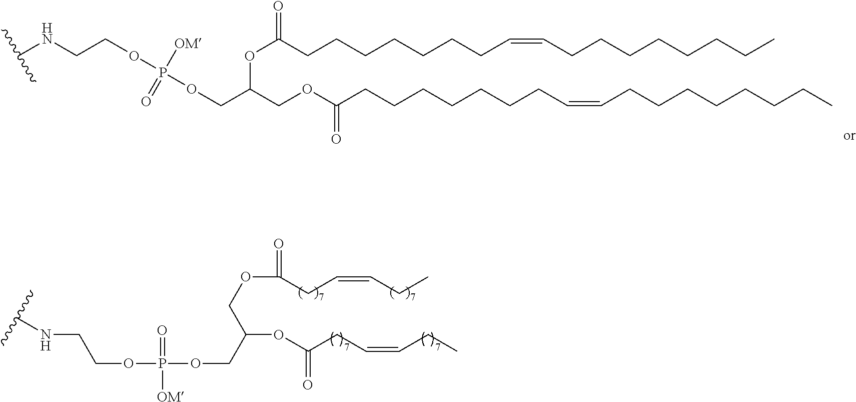

where M′ is a monovalent cation (typically H⁺).

The invention will now be described with reference to embodiments or examples and the figures of the accompanying drawings pages.

### BRIEF DESCRIPTION OF DRAWINGS

FIG. 1. ¹H NMR spectrum of the cyanoselenide-lipid construct designated NCSeCH₂CO—CMG(2)-Ad-DOPE

FIG. 2. Fluorescence microscopy of the surface of untreated (A) and treated (B) coupons following incubation in the presence of viable cultures of Staphylococcus aureus.

FIG. 3. Fluorescence microscopy of the surface of untreated (A) and treated (B) coupons following incubation in the presence of viable cultures of Staphylococcus epidermis.

FIG. 4. Photographs of incubated blood agar plates following inoculation with cultures of Staphylococcus aureus exposed to untreated (A) and treated (B) coupons.

FIG. 5. Photographs of incubated blood agar plates following inoculation with cultures of Staphylococcus epidermis exposed to untreated (A) and treated (B) coupons.

FIG. 6. Scanning electron micrographs (350×) of samples of untreated (A) and treated (B) surgical dressing using the construct designated NCSeCH₂CO—CMG(2)-Ad-DOPE in the treatment.

FIG. 7. Scanning electron micrographs (3,500×) of samples of untreated (A) and treated (B) surgical dressing using the construct designated NCSeCH₂CO—CMG(2)-Ad-DOPE in the treatment.

FIG. 8. Scanning electron micrographs (4,500×) of surfaces of stainless steel squares treated with the construct designated NCSeCH₂CO—CMG(2)-Ad-DOPE (A), the construct designated HA-gar-Ad-DOPE (B), the construct designated Spm-Ad-DOPE (C) or untreated (D) and incubated in the presence of Staphylococcus aureus.

FIG. 9. Scanning electron micrographs (4,500×) of surfaces of stainless steel squares treated with the construct designated NCSeCH₂CO—CMG(2)-Ad-DOPE (A), the construct designated HA-gar-Ad-DOPE (B), the construct designated Spm-Ad-DOPE (C) or untreated (D) and incubated in the presence of Staphylococcus epidermis.

FIG. 10. Scanning electron micrographs at the magnifications indicated in each micrograph of the surface of Capture-R™ Ready-Id (A), Capture-R™ Ready-Screen (B), CT-6 (C), Spm-Ad-DOPE (9a) treated and fixed (D), Spm-Ad-DOPE (9a) treated and lysed (E) and untreated (F) plates.

FIG. 11. Photomicrographs (100×) of surfaces treated with Spm-Ad-DOPE (9a) in either PBS (A) or SucT (B) or spermine (C) following contact with a suspension of RBCs.

FIG. 12. Scanning electron micrographs of the surface of untreated (A) and treated (B) laminated nylon mesh following exposure to particulates generated by a wood burner.

### DESCRIPTION OF EMBODIMENTS

The method of the invention provides a convenient biocompatible means of treating surgical dressings and implants at the location and time of use by clinicians and surgeons.

### Cyanoselenide as the Functional Moiety

The preparation of the constructs designated Mal-(CH₂)₂CO—CMG(2)-Ad-DOPE and H—CMG(2)-Ad-DOPE is disclosed in the publication of Bovin et al (2008) and restated here for the sake of completeness. Acetone, benzene, chloroform, ethylacetate, methanol, toluene and o-xylene were from Chimmed (Russian Federation). Acetonitrile was from Cryochrom (Russian Federation). DMSO, DMF, CF₃COOH, Et₃N, N,N′-dicyclohexylcarbodiimide and N-hydroxysuccinimide were from Merck (Germany). Iminodiacetic acid dimethyl ester hydrochloride was from Reakhim (Russian Federation). Dowex 50X4-400 and Sephadex LH-20 were from Amersham Biosciences AB (Sweden). Silica gel 60 was from Merck (Germany). Tetraamine (H₂N—CH₂)₄C x 2H₂SO₄ was synthesized as described by Litherland et al. (1938). Thin-layer chromatography was performed using silica gel 60 F₂₅₄ aluminium sheets (Merck, 1.05554) with detection by charring after 7% H₃PO₄ soaking.

Preparation of {[2-(2-tert-butoxycarbonylamino-acetylamino)-acetyl]-methoxycarbonylmethyl -amino}-acetic acid methyl ester

To a stirred solution of (methoxycarbonylmethyl-amino)-acetic acid methyl ester hydrochloride (988 mg, 5 mmol) in DMF (15 ml) were added Boc-GlyGlyNos (3293 mg, 10 mmol) and (CH₃CH₂)₃N (3475 μL, 25 mmol) were added. The mixture was stirred overnight at room temperature and then diluted with o-xylene (70 ml) and evaporated. Flash column chromatography on silica gel (packed in toluene, and eluted with ethyl acetate) resulted in a crude product. The crude product was dissolved in chloroform and washed sequentially with water, 0.5 M NaHCO₃ and saturated KCl. The chloroform extract was evaporated and the product purified on a silica gel column (packed in chloroform and eluted with 15:1 (v/v) chloroform/methanol). Evaporation of the fractions and drying under vacuum of the residue provided a colourless thick syrup. Yield 1785 mg, (95%). TLC: R_{f}=0.49 (7:1 (v/v) chloroform/methanol).

¹H NMR (500 MHz, [D₆]DMSO, 30° C.) δ, ppm: 7.826 (t, J=5.1 Hz, 1H; NHCO), 6.979 (t, J=5.9 Hz, 1H; NHCOO), 4.348 and 4.095 (s, 2H; NCH₂COO), 3.969 (d, J=5.1 Hz, 2H; COCH₂NH), 3.689 and 3.621 (s, 3H; OCH₃), 3.559 (d, J=5.9 Hz, 2H; COCH₂NHCOO), 1.380 (s, 9H; C(CH₃)₃).

Preparation of {[2-(2-tert-butoxycarbonylamino-acetylamino)-acetyl]-methoxycarbonylmethyl -amino}-acetic acid

To a stirred solution of {[2-(2-tert-butoxycarbonylamino-acetylamino)-acetyl]-methoxycarbonylmethyl-amino}-acetic acid methyl ester (1760 mg, 4.69 mmol) in methanol (25 ml) 0.2 M aqueous NaOH (23.5 ml) was added and the solution kept for 5 min at room temperature. The solution was then acidified with acetic acid (0.6 ml) and evaporated to dryness. Column chromatography of the residue on silica gel (packed in ethyl acetate and eluted with 2:3:1 (v/v/v) i-PrOH/ethyl acetate/water) resulted in a recovered {[2-(2-tert-butoxycarbonylamino -acetylamino)-acetyl]-methoxycarbonylmethyl-amino}-acetic acid methyl ester (63 mg, 3.4%) and target compound (1320 mg). The intermediate product was then dissolved in methanol/water/pyridine mixture (20:10:1, 30 ml) and passed through an ion exchange column (Dowex 50X4-400, pyridine form, 5 ml) to remove residual sodium cations. The column was then washed with the same solvent mixture, the eluant evaporated, the residue dissolved in chloroform/benzene mixture (1:1, 50 ml) and then evaporated and dried under vacuum. Yield of 10 was 1250 mg (74%), white solid. TLC: R_{f}=0.47 (4:3:1 (v/v/v) i-PrOH/ethyl acetate/water).

¹H NMR (500 MHz, [D₆]DMSO, 30° C.), mixture of cis- and trans-conformers of N-carboxymethylglycine unit c.3:1. Major conformer; δ, ppm: 7.717 (t, J=5 Hz, 1H; NHCO), 7.024 (t, J=5.9 Hz, 1H; NHCOO), 4.051 (s, 2H; NCH₂COOCH₃), 3.928 (d, J=5 Hz, 2H; COCH₂NH), 3.786 (s, 2H; NCH₂COOH), 3.616 (s, 3H; OCH₃), 3.563 (d, J=5.9 Hz, 2H; COCH₂NHCOO), 1.381 (s, 9H; C(CH₃)₃) ppm; minor conformer, δ=7.766 (t, J=5 Hz, 1H; NHCO), 7.015 (t, J=5.9 Hz, 1H; NHCOO), 4.288 (s, 2H; NCH₂COOCH₃), 3.928 (d, J=5 Hz, 2H; COCH₂NH), 3.858 (s, 2H; NCH₂COOH), 3.676 (s, 3H; OCH₃), 3.563 (d, J=5.9 Hz, 2H; COCH2NHCOO), 1.381 (s, 9H; C(CH₃)₃).

Preparation of {[-2-(2-tert-butoxycarbonylamino-acetylamino)-acetyl]-methoxycarbonylmethyl -amino}-acetic acid N-oxysuccinimide ester (Boc-Gly₂(MCMGly) Nos)

To an ice-cooled stirred solution of {[2-(2-tert-butoxycarbonylamino-acetylamino) -acetyl]-methoxycarbonylmethyl-amino}-acetic acid (1200 mg, 3.32 mmol) and N-hydroxysuccinimide (420 mg, 3.65 mmol) in DMF (10 ml) was added N,N′-dicyclohexylcarbodiimide (754 mg, 3.65 mmol). The mixture was stirred at 0° C. for 30 min, then for 2 hours at room temperature. The precipitate of N,N′-dicyclohexylurea was filtered off, washed with DMF (5 ml), and filtrates evaporated to a minimal volume. The residue was then agitated with (CH₃CH₂)₂O (50 ml) for 1 hour and an ether extract removed by decantation. The residue was dried under vacuum providing the active ester (1400 mg, 92%) as a white foam. TLC: R_{f}=0.71 (40:1 (v/v) acetone/acetic acid).

¹H NMR (500 MHz, [D₆]DMSO, 30° C.), mixture of cis- and trans-conformers of N-carboxymethylglycine unit c. 3:2.

Major conformer; δ, ppm: 7.896 (t, J=5.1 Hz, 1H; NHCO), 6.972 (t, J=5.9 Hz, 1H; NHCOO), 4.533 (s, 2H; NCH₂COON), 4.399 (s, 2H; NCH₂COOCH₃), 3.997 (d, J=5.1 Hz, 2H; COCH₂NH), 3.695 (s, 3H; OCH₃), 3.566 (d, J=5.9 Hz, 2H; COCH₂NHCOO), 1.380 (s, 9H; C(CH₃)₃).

Minor conformer; δ, ppm: 7.882 (t, J=5.1 Hz, 1H; NHCO), 6.963 (t, J=5.9 Hz, 1H; NHCOO), 4.924 (s, 2H; NCH₂COON), 4.133 (s, 2H; NCH₂COOCH₃), 4.034 (d, J=5.1 Hz, 2H; COCH₂NH), 3.632 (s, 3H; OCH₃), 3.572 (d, J=5.9 Hz, 2H; COCH₂NHCOO), 1.380 (s, 9H; C(CH₃)₃).

The active ester (1380 mg) was dissolved in DMSO to provide a volume of 6 ml and used as a 0.5 M solution (stored at −18° C.).

Preparation of {[2-(2-tert-butoxycarbonylamino-acetylamino)-acetyl]-methoxycarbonylmethyl -amino}-acetic acid methyl ester

To the stirred solution of (methoxycarbonylmethyl-amino)-acetic acid methyl ester hydrochloride (988 mg, 5 mmol) in DMF (15 ml) Boc-GlyGlyNos (3293 mg, 10 mmol) and Et₃N (3475 μl, 25 mmol) were added. The mixture was stirred overnight at room temperature (r.t.), then diluted with o-xylene (70 ml) and evaporated. Flash column chromatography on silica gel (packed in toluene and eluted with ethyl acetate) resulted in crude product. The crude product was dissolved in chloroform and washed sequentially with water, 0.5 M NaHCO₃ and saturated KCl. The chloroform extract was evaporated, and the product was purified on a silica gel column (packed in chloroform and eluted with chloroform/methanol 15:1). Evaporation of fractions and vacuum drying of residue resulted in a colorless thick syrup of (3) (1785 mg, 95%). TLC: R_{f}=0.49 (chloroform/methanol 7:1).

¹H NMR (500 MHz, [D₆]DMSO, 30° C.) δ=7.826 (t, J=5.1 Hz, 1H; NHCO), 6.979 (t, J=5.9 Hz, 1H; NHCOO), 4.348 and 4.095 (s, 2H; NCH₂COO), 3.969 (d, J=5.1 Hz, 2H; COCH₂NH), 3.689 and 3.621 (s, 3H; OCH₃), 3.559 (d, J=5.9 Hz, 2H; COCH₂NHCOO), 1.380 (s, 9H; CMe₃) ppm.

Preparation of {[2-(2-tert-butoxycarbonylamino-acetylamino)-acetyl]-methoxycarbonylmethyl -amino}-acetic acid

To the stirred solution of {[2-(2-tert-butoxycarbonylamino-acetylamino)-acetyl]-methoxycarbonylmethyl-amino}-acetic acid methyl ester (1760 mg, 4.69 mmol) in methanol (25 ml) 0.2 M aqueous NaOH (23.5 ml) was added. The solution was kept for 5 min at r.t., then acidified with acetic acid (0.6 ml) and evaporated to dryness. Column chromatography of the residue on silica gel (packed in ethyl acetate and eluted with iPrOH/ethyl acetate/water (2:3:1)) resulted in recovered (3) (63 mg, 3.4%) and crude target compound (1320 mg). The crude target compound was dissolved in methanol/water/pyridine mixture (20:10:1, 30 ml) and passed through an ion-exchange column (Dowex 50X4-400, pyridine form, 5 ml) to remove residual Na cations. The column was washed with the same mixture, eluant evaporated, dissolved in chloroform/benzene mixture (1:1, 50 ml) then evaporated and dried in vacuum to provide a yield of pure (10) was 1250 mg (74%), white solid. TLC: R_{f}=0.47 (iPrOH/ethyl acetate/water (4:3:1)).

¹H NMR (500 MHz, [D₆]DMSO, 30° C.) of mixture of cis- and trans-conformers of N-carboxymethyl-glycine unit c.3:1.

Major conformer: δ=7.717 (t, J=5 Hz, 1H; NHCO), 7.024 (t, J=5.9 Hz, 1H; NHCOO), 4.051 (s, 2H; NCH₂COOMe), 3.928 (d, J=5 Hz, 2H; COCH₂NH), 3.786 (s, 2H; NCH₂COOH), 3.616 (s, 3H; OCH₃), 3.563 (d, J=5.9 Hz, 2H; COCH₂NHCOO), 1.381 (s, 9H; CMe₃) ppm.

Minor conformer: δ=7.766 (t, J=5 Hz, 1H; NHCO), 7.015 (t, J=5.9 Hz, 1H; NHCOO), 4.288 (s, 2H; NCH₂COOMe), 3.928 (d, J=5 Hz, 2H; COCH₂NH), 3.858 (s, 2H; NCH₂COOH), 3.676 (s, 3H; OCH₃), 3.563 (d, J=5.9 Hz, 2H; COCH₂NHCOO), 1.381 (s, 9H; CMe₃) ppm.

Preparation of {[2-(2-tert-butoxycarbonylamino-acetylamino)-acetyl]-methoxycarbonylmethyl -amino}-acetic acid N-oxysuccinimide ester Boc-Gly2 (MCMGly) Nos

To an ice-cooled stirred solution of {[2-(2-tert-butoxycarbonylamino-acetylamino) -acetyl]-methoxycarbonylmethyl-amino}-acetic acid (1200 mg, 3.32 mmol) and N-hydroxysuccinimide (420 mg, 3.65 mmol) in DMF (10 ml) N,N′-dicyclohexylcarbodiimide (754 mg, 3.65 mmol) was added. The mixture was stirred at 0° C. for 30 min, then for 2 h at r.t. The precipitate of N,N′-dicyclohexylurea was filtered off, washed with DMF (5 ml) and the filtrates evaporated to a minimal volume. The residue was agitated with Et₂O (50 ml) for 1 h. An ether extract was removed by decantation, and the residue dried in vacuum to yield the target compound (1400 mg, 92%) as a white foam. TLC: R_{f}=0.71 (acetone/acetic acid 40:1).

¹H NMR (500 MHz, [D₆]DMSO, 30° C.), mixture of cis- and trans-conformers of N-carboxymethyl -glycine unit c. 3:2.

Major conformer: δ=7.896 (t, J=5.1 Hz, 1H; NHCO), 6.972 (t, J=5.9 Hz, 1H; NHCOO), 4.533 (s, 2H; NCH₂COON), 4.399 (s, 2H; NCH₂COOMe), 3.997 (d, J=5.1 Hz, 2H; COCH₂NH), 3.695 (s, 3H; OCH₃), 3.566 (d, J=5.9 Hz, 2H; COCH₂NHCOO), 1.380 (s, 9H; CMe₃) ppm.

Minor conformer: δ=7.882 (t, J=5.1 Hz, 1H; NHCO), 6.963 (t, J=5.9 Hz, 1H; NHCOO), 4.924 (s, 2H; NCH₂COON), 4.133 (s, 2H; NCH₂COOMe), 4.034 (d, J=5.1 Hz, 2H; COCH₂NH), 3.632 (s, 3H; OCH₃), 3.572 (d, J=5.9 Hz, 2H; COCH₂NHCOO), 1.380 (s, 9H; CMe₃) ppm.

Preparation of the Constructs Designated Mal-(CH₂)₂CO—CMG(2)-Ad-DOPE and H—CMG (2) -Ad-DOPE

The construct designated H—CMG(2)-Ad-DOPE was prepared from {[2-(2-tert-butoxycarbonylamino -acetylamino)-acetyl]-methoxycarbonylmethyl-amino}-acetic acid N-oxysuccinimide ester Boc-Gly₂(MCMGly)Nos according to Scheme III of the publication of Bovin et al (2008). The construct designated Mal-(CH₂)₂CO—CMG(2)-Ad-DOPE was prepared according to the first step of Scheme IV of the publication of Bovin et al (2008). Briefly, the construct designated H—CMG(2)-Ad-DOPE was treated with a 5-fold excess of 3-maleimidopropionic acid oxybenztriazol ester in i-PrOH-water. The maleimide-lipid construct was isolated in 40% yield after gel-permeation chromatography on Sephadex LH-20 (i-PrOH-water, 1:2).

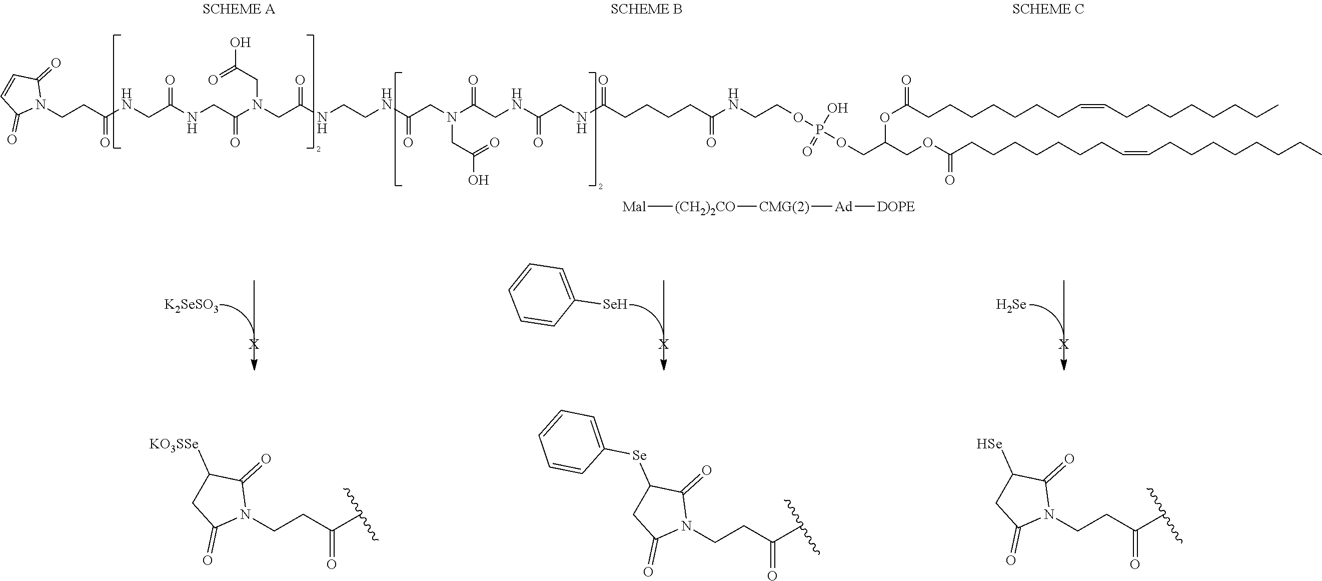

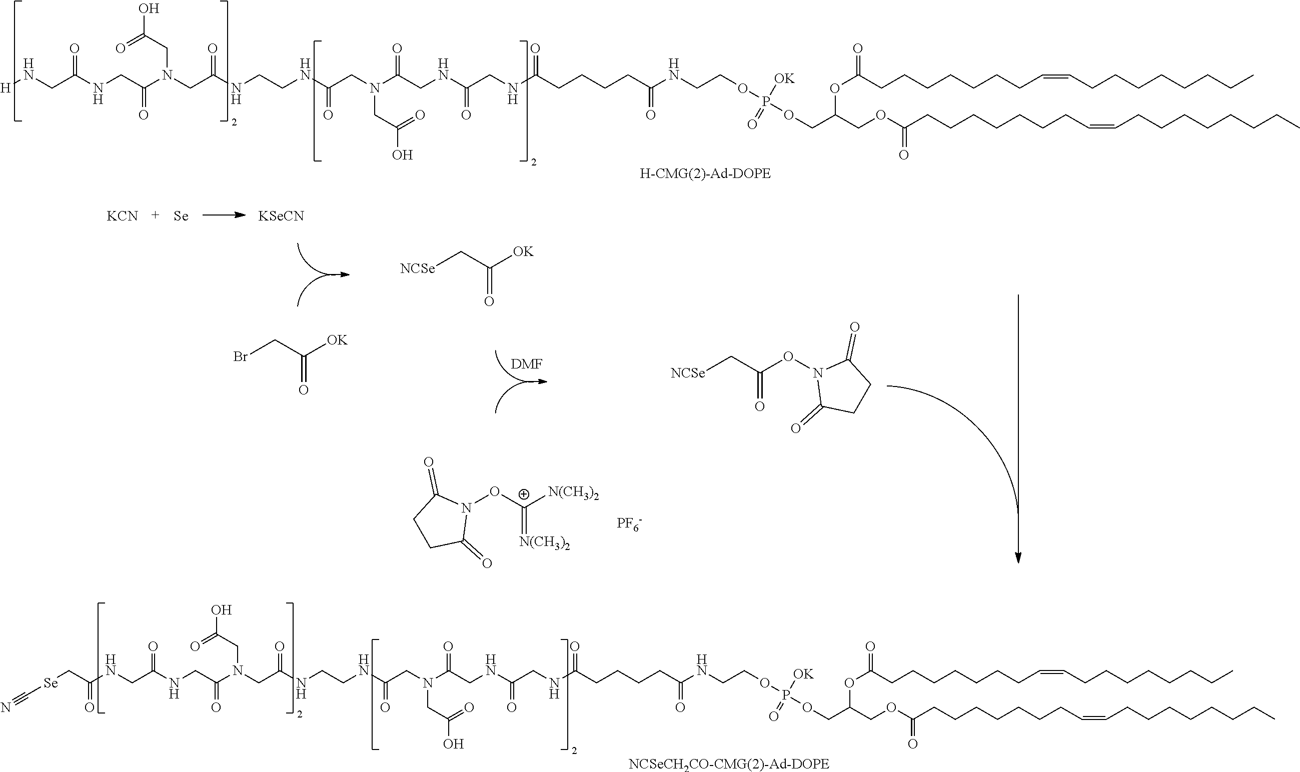

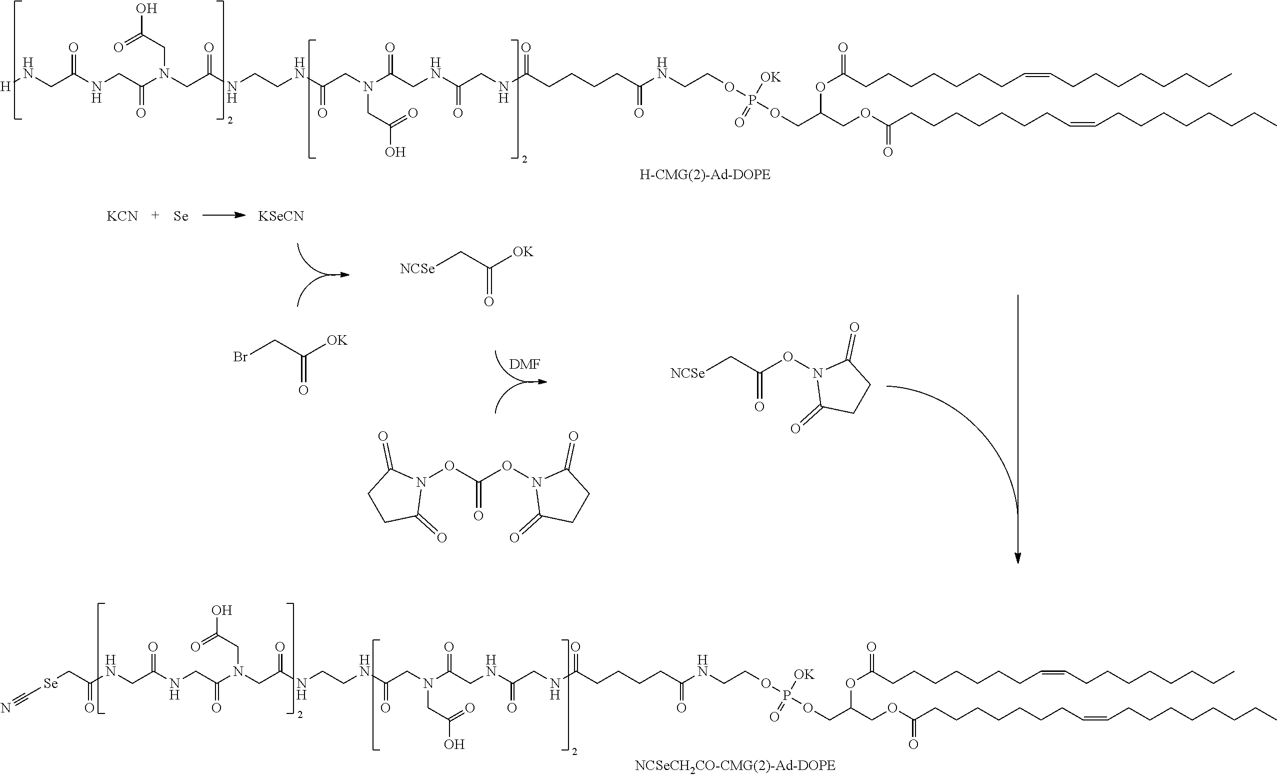

Preparation of NCSeCH₂CO—CMG(2)-Ad-DOPE

Attempts to prepare a cyanoselenide-lipid construct via an addition reaction between the maleimide-lipid construct designated Mal-(CH₂)₂CO—CMG(2)-Ad-DOPE and potassium selenosulfite (K₂SeSo₃) [SCHEME A], selenophenol (PhSeH) [SCHEME B] and hydrogen selenide (H₂Se) [SCHEME C] were unsuccessful. With hindsight the failure to obtain a stable seleno-Bunte salt according to SCHEME A is at least in part predictable from the disclosure of the chemical behaviour of their sulfur analogues in the publication of Distler (1967). Both the attempted Michael additions of phenylselenide and hydrogen selenide in protic media according to SCHEME B and SCHEME C, respectively, yielded a product with a reduced maleimide double bond, as opposed to the desired selenylsuccinimides. Formation of selenylsuccinimides in quantitative yield has been disclosed in the publication of Numeo et al (1981). However, the disclosed use of anhydrous ether is incompatible with the use of the polyanionic maleimide-lipid construct designated Mal-(CH₂)₂CO—CMG(2)-Ad-DOPE.

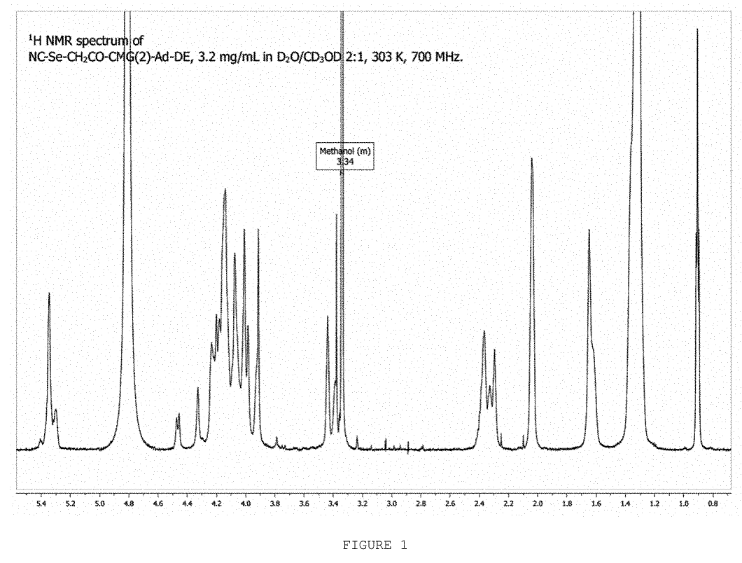

It was subsequently discovered that the cyanoselenide-lipid construct designated NCSeCH₂CO—CMG(2)-Ad-DOPE could be successfully prepared via an activated 2-selenocyanatoacetic acid (NC—Se—CH₂COOH). The activated NC—Se—CH₂COOH was reacted with the lipid construct H—CMG(2)-Ad-DOPE according to SCHEME D(a) or SCHEME D(b). The prepared construct was stored in the dark under an inert atmosphere. Potassium selenocyanate was selected as the reagent of choice as it could readily be activated as an N-hydroxysuccinimide (NHS) ester according to SCHEME D(a) or (b) or mixed anhydride according to SCHEME D(c). Potassium selenocyanoacetate (NCSeCH₂COOK) was synthesized from freshly prepared solutions of potassium selenocyanate (KSeCN) and potassium bromoacetate (BrCH₂COOK) according to the procedures disclosed in the publication of Klauss (1970). The synthesized NCSeCH₂COOK was stored in a vacuum desiccator over potassium hydroxide (KOH) pellets in the dark prior to activation. For activation the potassium selenocyanoacetate (156 mg, 0.77 mmol) was added in one portion to a solution of N,N,N′,N′-tetramethyl-O-(N-succinimidyl)uraniumhexafluorophosphate (HSTU) (IRIS, Germany) (212 mg, 0.59 mmol) in 1 mL DMF while a gentle flow of dry argon via a PTFE capillary was bubbling through. The slurry thus obtained was stirred in this way for 30 minutes during which the initial solid changed to a more dense crystalline precipitate (KPF₆). The reaction mixture was sonicated for 1 to 2 minutes and combined with the construct designated H-CMG(2)-Ad-DOPE (110 mg, 0.06 mmol) dissolved in 1 mL of 20% IPA followed by 100 μL 1N KHCO₃. A sticky solid (presumably NCSeCH₂COOSu) that precipitated immediately, was dissolved by dropwise addition of 30% IPA (circa 1.6 mL) with sonication and the reaction mixture was magnetically stirred for 3 hours at room temperature keeping pH in the range 8.0 to 8.5 (TLC control: Solvents were evaporated in vacuum and dry residue was triturated with 3 mL of acetonitrile with sonication until fine slurry formed and then transferred into Eppendorf tubes (2×2.2 mL), centrifuged and the solids washed 4 times consecutively with neat IPA and MeCN (2 mL of each, brief sonication followed by centrifugation). The wet solids were dissolved in 3.5 mL of 30% IPA-water and lyophilized to constant weight. 111 mg (92%) of the cyanoselenide-lipid construct designated NCSeCH₂CO—CMG(2)-Ad-DOPE were obtained as a reddish amorphous powder. R_{f}˜0.5, CHCl₃/methanol/water 2:6:1 (v/v); TLC aluminium sheets Silica gel 60 F₂₅₄ (Merck 1.05554). It is noted that mass spectroscopy did not appear suitable for the characterization of this construct. Only peaks of Se-free fragments could be detected. The 1H NMR spectrum determined for the construct is provided in FIG. 1.

### Polycation as the Functional Moiety

The polycation lipid construct 9a was prepared and isolated as its trifluoroacetic acid (TFA) salt (SCHEME E). Briefly, desymmetritisation of the polyamine spermine [CAS # 71-44-3](2) was performed according to a modified version of the method disclosed in the publication of Geall and Blagbrough (2000) employing Boc as the protecting group. It will be recognised that the method is also applicable to the desymmetritisation of other unbranched polyamines such as spermidine [CAS # 124-20-9](1), tetraethylenepentamine [CAS # 112-57-2] (3); pentaethylenehexamine [CAS # 4067-16-7] (4) and hexaethyleneheptamine [4403-32-1](5). Accordingly, a series of polycation lipid constructs may be accessed according to SCHEME E.

According to SCHEME E the Boc protected, desymmetritised intermediate N¹,N⁴,N⁹-tri-tert-butoxycarbonyl)-1,12-diamino-4,9-diazadodecane (6) is conjugated to the diacylglycerophospholipid 1,2-O-dioleoyl-sn-glycero-3-phosphatidyl -ethanolamine [CAS # 4004-05-1] (DOPE) using the homobifunctional crosslinker disuccinimidyl adipate. It will be recognised that other disuccinimidyl compounds may be employed as the homobifunctional crosslinker. These include

The activated lipid (7a) acylates the terminal, primary amino group of N¹,N⁴,N⁹-tri-tert-butoxycarbonyl)-1,12-diamino-4,9-diazadodecane (6) to provide a lipidated Boc protected polyamine intermediate (8a). Again, it will be recognised that according to Scheme I other diacylglycerophospholipids, such as 1,2-O-distereoyl-sn-glycero-3-phosphatidylethanolamine [CAS # ] (DSPE) may be substituted for DOPE.

In the final step of SCHEME E the lipidated polyamine intermediate (8a) is deprotected and the polycation lipid construct (9a) isolated as its trifluoroacetic acid salt.

Materials and Methods

Chloroform, dichloroethane, dichloromethane, methanol and toluene were obtained from Chimmed (Russian Federation). Trifluoroacetic acid, triethylamine, di-tert-butyldicarbonate methyl trifluoroacetate were obtained from Merck (Germany). Spermine was obtained from Sigma-Aldrich (USA). Sephadex LH-20 was obtained from Amersham Biosciences AB (Sweden). Silica gel 60 was obtained from Merck (Germany). Thin layer chromatographic (TLC) analysis was performed on silica gel 60 F₂₅₄ plates (Merck). Amino containing compounds were detected using ninhydrin reagent. DOPE containing compounds were detected using an aqueous solution of potassium permanganate (KMnO₄) or by soaking in 8% (w/v) phosphoric acid in water followed by heating at over 200° C. ¹H NMR spectra were recorded at 30° C. with a Bruker BioSpin GmbH 700 MHz instrument using the signal of the solvent's residual protons as reference ([D]CHCl₃, 7.270 ppm; [D2]H₂O, 4.750 ppm). Mass spectra were recorded with an Agilent ESI-TOF 6224 LC/MS spectrometer.

Preparation of Boc₃Spm (6)

To a stirred solution of spermine (2) (1 equivalent, 1.34 g, 6.6 mmol) in methanol (90 mL) at −80° C. under nitrogen, a solution of methyl trifluoroacetate (1.1 equivalents, 0.730 mL, 7.26 mmol) in methanol (1.5 mL) was added drop-wise over a period of 30 min. Stirring was continued at −80° C. for a further period of 30 min and then the temperature increased to 0° C. The reaction afforded predominantly the mono-trifluoroacetamide. Without isolation, the remaining amino functional groups were quantitatively protected by drop-wise addition of an excess of di-tert-butyldicarbonate (4 equivalents, 5.76 g, 26.4 mmol) in methanol over a period of 3 min. The reaction was then warmed to 25° C. and stirred for a further 15 hr to afford the fully protected spermine (R_{f} 0.33 (95:5 (v/v) CHCl₃-i-PrOH)). The trifluoroacetate protecting group was then removed in situ by increasing the pH of the solution to greater than 11 pH units with concentrated aqueous ammonia (conc. aq. NH₃) and then stirred at 25° C. for a period of 15 hr. The solution was concentrated in vacuo and the residue purified over silica gel (95:5:1 to 90:10:1 (v/v/v) CHCl₃-MeOH-conc. aq. NH₃) to afford the title compound (6) as a colourless homogeneous oil (1.5 g, 45%), R_{f} 0.32 (83:16:1 (v/v/v) CHCl₃-MeOH-conc. aq. NH₃). MS, m/z: found 502.3725 (M⁺+1), C₂₅H₅₀N₄O₆ required M⁺ 501.3652.

¹H-NMR (700 MHz, CDCl₃, 303° K.), δ, ppm: 3.4 (m, 2H, 1—CH₂), 3.05-3.30 (m, 8H, 3,4,7,8-CH₂), 3.01(m, 2H, 10-CH₂), 2.03 (m, 2H, 9-CH₂), 1.67 (m, 2H, 2-CH₂), 1.50 (m, 4H, 5,6-CH₂), 1.44, 1.45, 1.46 (3 s, overlapping, 27H, 3 O—C(CH₃)₃).

Preparation of SuO-Ad-DOPE (7a) and SuO-Ad-DSPE (7b)

To a solution of disuccinimidyl adipate (70 mg, 205 μmol) in dry N,N-dimethylformamide (1.5 ml) were added DOPE or DSPE (40 μmol) in chloroform (1.5 ml) followed by triethylamine (7 μl). The mixture was kept for 2 h at room temperature, then neutralized with acetic acid and partially concentrated in vacuo. Column chromatography (Sephadex LH-20, 1:1 (v/v) chloroform-methanol, 0.2% (w/v) aqueous acetic acid) of the residue yielded SuO-Ad-DOPE (7a) (37 mg, 95%) as a colourless syrup. TLC (6:3:0.5 (v/v/v) chloroform-methanol-water) R_{f} 0.5 (SuO-Ad-DOPE (7a)) and R_{f} 0.55 (SuO-Ad-DOPE (7b)).

¹H NMR (2:1 (v/v) CDCl₃/CD₃OD) δ:

SuO-Ad-DOPE (7a) −5.5 (m, 4H, 2x(—CH═CH—), 5.39 (m, 1H, —OCH₂—CHO—CH₂O—), 4.58 (dd, 1H, J=3.67, J=11.98, —CCOOHCH—CHO—CH₂O—), 4.34 (dd, 1H, J=6.61, J=11.98, —CCOOHCH—CHO—CH₂O—), 4.26 (m, 2H, PO—CH₂—CH₂—NH₂), 4.18 (m, 2H, —CH₂—OP), 3.62 (m, 2H, PO—CH₂—CH₂—NH₂), 3.00 (s, 4H, ONSuc), 2.8 (m, 2H, —CH₂—CO (Ad), 2.50 (m, 4H, 2x(—CH₂—CO), 2.42 (m, 2H, —CH₂-00 (Ad), 2.17 (m, 8H, 2x(—CH₂—CH═CH—CH₂—), 1.93 (m, 4H, COCH₂CH₂CH₂CH₂CO), 1.78 (m, 4H, 2x(COCH₂cH₂—), 1.43, 1.47 (2 bs, 40H, 20 CH₂), 1.04 (m, 6H, 2 CH₃).

SuO-Ad-DSPE (7b) −5.39 (m, 1H, —OCH₂—CHO—CH₂O—), 4.53 (dd, 1H, J=3.42, J=11.98, —CCOOHCH—CHO—CH₂O—), 4.33 (dd, 1H, J=6.87, J=11.98, —CCOOHCH—CHO—CH₂O—), 4.23 (m, 2H, PO—CHH₂—CH₂—NH₂), 4.15 (m, 2H, —CH₂—OP), 3.61 (m, 2H, PO—CH₂—CH₂—NH₂), 3.00 (s, 4H, ONSuc), 2.81 (m, 2H, —CH₂—CO (Ad), 2.48 (m, 4H, 2x(—CH₂—CO), 2.42 (m, 2H, —CH₂—CO (Ad), 1.93 (m, 4H, COCH₂CH₂CH₂CH₂CO), 1.78 (m, 4H, 2x(COCH₂CH₂—), 1.43, 1.47 (2 bs, 40H, 20 CH₂), 1.04 (m, 6H, 2 CH₃).

Preparation of Boc₃Spm-Ad-DOPE (8a)

To a stirred solution of Boc₃Spm (6) (552 mg, 1.1 mmol) in dichloroethane (25 ml) was added trimethylamine (1 ml, 7.2 mmol) followed by a solution of SuO-Ad-DOPE (1066 mg, 1.1 mmol) in dichloroethane (25 ml). The reaction mixture was stirred for a period of 2 hr and then the solvent was removed under reduced pressure at 37° C. The crude product was purified by chromatography on silica gel by elution with 97:3 to 85:15 (v/v) CHCl₃—MeOH to afford the title compound (8a) (1.16 g, 78%) as a viscous oil. TLC (10:6:0.8 (v/v/v) CH₂Cl₂—EtOH—H₂O) R_{f} 0.36.

¹H NMR (700 MHz, CDCl₃/CD₃OD 1:1, 10 mg/mL, 303° K.) δ, ppm: 5.34 (m, 4H; 2 CHH═CHH), 5.19 (m, 1H; OCH₂CHCH₂O), 4.37 (dd, J_{gem}˜11.1 Hz, 1H, POCH₂—CH—CH^{a}—O(CO)), 4.13 (dd, J˜7.2 Hz, 1H, POCH₂—CH—CH^{b}—O(CO)), 3.94 (m, 4H), 3.48 (m, 2H), 3.05-3.30 (m, 12H, 1,3,4,7,8,10-CH₂), 2.71 (m, 2H), 2.20-2.42 (m, 8H), 1.98-2.04 (m, 8H), 1.64 (m, 8H,), 1.58 (m, 4H), 1.49 (m, 4H, 5,6-CH₂), 1.44, 1.45, 1.46 (3s, 27H, 3 O—C(CH₃)₃), 1.22-1.37 (m, 40H, 20 CH₂), 0.88 and 0.89 (2d, J≈7 Hz, 6H, 2 CH₃).

Preparation of Spm-Ad-DOPE (9a)

To a stirred solution of 8a (1.16 g, 0.85 mmol) in CHCl₃ (10 ml) at 25° C. TFA (5 ml, 95%) was added. After a period of 20 min the solution was concentrated in vacuo at 35° C. and the residue was co-evaporated with toluene (5 times 10 mL) to remove trace amounts of TFA. To remove any low molecular weight impurities the residue was dissolved in 1:1 (v/v) CHCl₃—MeOH (2 mL) and passed in two portions through a Sephadex LH-20 column (volume 330 mL, eluent 1:1 (v/v) CHCl₃—MeOH). Fractions containing pure 9a (di-TFA salt) were combined and evaporated to dryness and the residue dissolved in water (˜100 mL) and freeze-dried. A yield of was 975 mg (89%) was obtained. MS, m/z: found 1056.8063 (M⁺+1), C₅₇H₁₁₀N₅O₁₀P required M⁺ 1055.779.

¹H NMR (700 MHz, 1:1 (v/v) CDCl₃—CD₃OD, 10 mg/mL, 303° K.) δ, ppm: 5.51 (m, 4H; 2 CH═CH),5.42 (m, 1H; OCH₂CHCH₂O), 4.6 (dd, J_{gem}=12.1 Hz, J=2.81 Hz, 1H, POCH₂—CH—CH^{a}—O(CO)), 4.34 (dd, J=7.09 Hz, 1H, POCH₂—CH—CH^{b}—O(CO)), 4.14 (m, 2H, POCH₂CH₂N), 4.06 (m, 2H, POCH₂—CH—CH₂), 3.59 (m, 2H, OCH₂CH₂N), 3.49 (m, 2H, 1-CH₂), 3.11-3.28 (m, 10H, 3,4,7,8,10-CH₂), 2.42 and 2.51 (2m, 8H, 4 COCH₂), 2.26 (m, 2H, 2-CH₂), 2.19 (m, 8H, 2 CH₂CH═CHCH₂), 2.07 (m, 2H, 9-CH₂), 1.99 (m, 4H, 5,6-CH₂), 1.79 (m, 8H, 4 COCH₂CH₂), 1.40-1.54 (m, 40H, 20 CH₂), 1.05 and 1.06 (2t, J≈7 Hz, 6H, 2 CH₃).

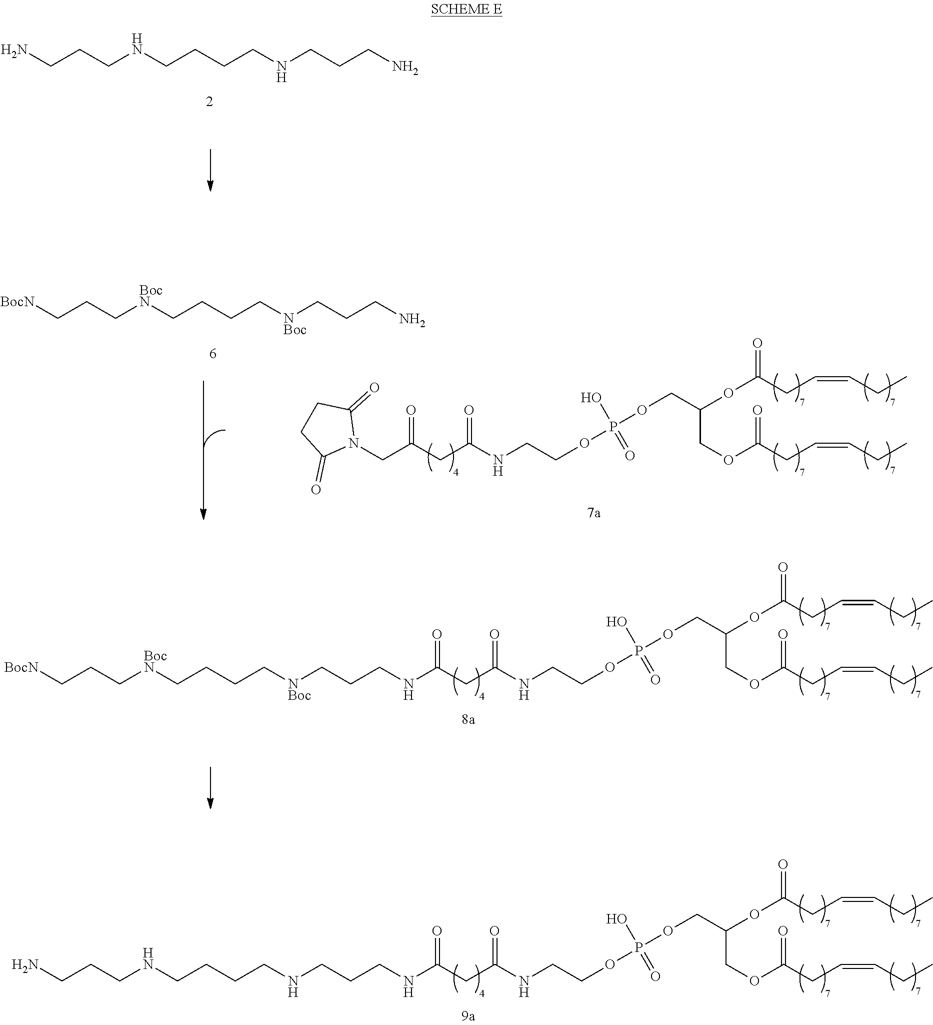

Surface Treatment—Antimicrobial

The ability of the cyanoselenide-lipid construct designated NCSeCH₂CO—CMG(2)-Ad-DOPE to prevent the growth of bacteria on the surface of stainless steel was evaluated. Used stainless steel (316 SS) coupons (catalogue no. RD123-316, Biosurface Technologies) were soaked in a 1% (v/v) aqueous solution of commercially available disinfectant cleaner (TRIGENE™) followed by soaking in a 0.1% (v/v) aqueous solution of commercially available alkaline cleaning agent (PYRONEG™) before rinsing with deionised water. Organic residues and metal dust were removed from the rinsed coupons by soaking in 95% (v/v) ethanol followed by rinsing in the same solvent and then sonicating for 30 minutes in methanol. Finally the coupons were immersed in boiling methanol for 10 minutes before being dried at 90° C., wrapped and autoclaved at 121° C. for 20 minutes. Treated coupons were prepared by immersion of the sterilised coupons in a degassed 50 μg/mL aqueous dispersion of the cyanoselenide-lipid construct designated NCSeCH₂CO—CMG(2)-Ad-DOPE. The aqueous dispersion was prepared from a degassed stock solution of the construct prepared at a concentration of 1 mg/mL in sterile distilled water. Untreated coupons were prepared as controls by immersion of the sterilised coupons in sterile distilled water. Treated and untreated coupons were dried in a laminar flow cabinet. Frozen stock solutions of Staphylococcus aureus and Staphylococcus epidermis were thawed and used to streak inoculate blood agar plates before incubation at 37° C. overnight. Isolated colonies were suspended in 10 mL sterile water to provide an approximate cell density in suspension of 1×10⁸ c.f.u./mL and confirmed by viability counts for each suspension on blood agar plates (S. aureus, 1.15×10⁸ c.f.u./mL; S. epidermis, 1.27×10⁷ c.f.u./mL). Individual dried coupons were transferred to the wells of a sterile microplate and the surface of each coupon contacted with 10 μL of a suspension of cells of Staphylococcus sp. and the suspension allowed to dry (circa 20 minutes). A volume of 1 mL of 3 g/L tryptic soy broth was then introduced into the well so as to cover the coupon and the microplate covered and incubated at 37° C. for 21 hours with agitation at 150 rpm. Following incubation coupons were removed, washed with water and dried.

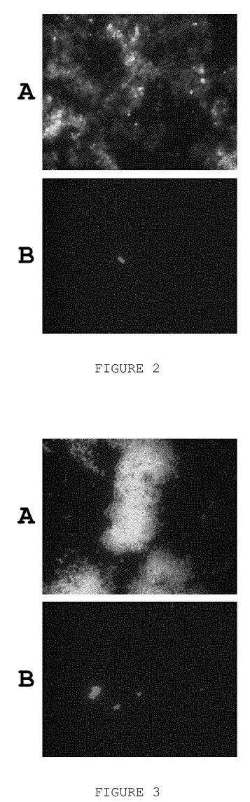

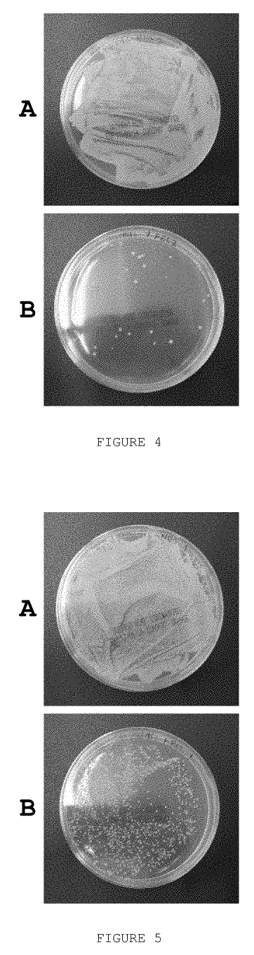

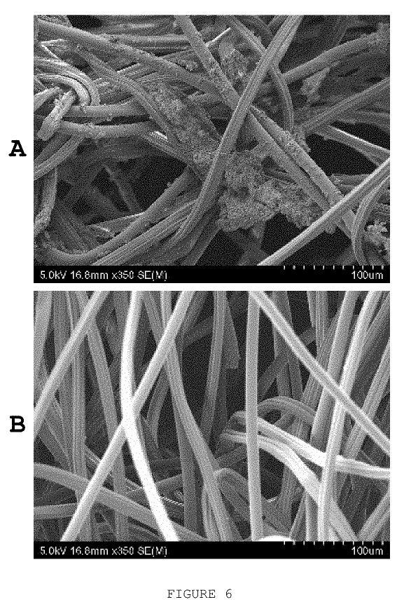

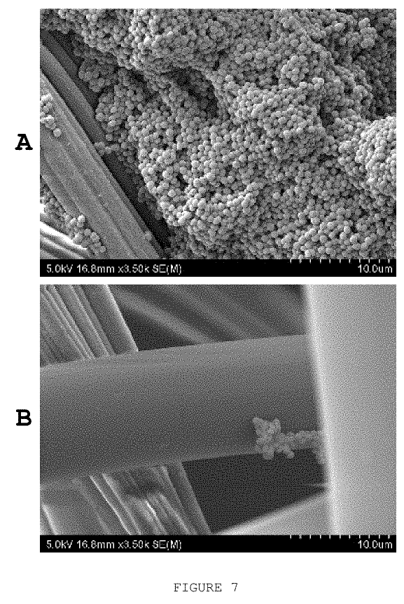

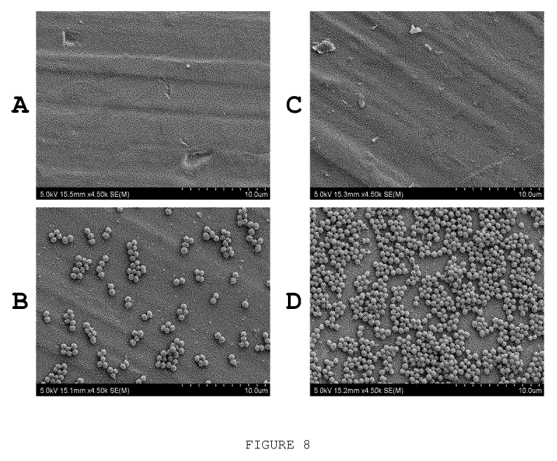

The surface of the dried coupons was then stained with acridine orange by placing three drops of the stain on the surface of each of the coupons for two minutes before rinsing with sterile water and air drying. The observations from fluorescence microscopy at 1,000× magnification are presented in FIG. 2 (S. aureus) and FIG. 3 (S. epidermis). Both species of Staphylococcus were able to establish biofilms on the surface of untreated coupons. Neither of the species was able to establish a biofilm on the treated coupons. To assess viability of bacteria following exposure to the coupons a 100 μL volume of the broth following incubation was spread on the surface of blood agar plates. The plates were then incubated at 37° C. overnight. Photographs of the incubated plates are presented in FIG. 4 (S. aureus) and FIG. 5 (S. epidermis). Exposure to the surface of the treated coupons significantly inhibited bacterial cell growth.

The ability of the polycation-lipid construct designated Spm-Ad-DOPE (9a) to prevent the growth of bacteria on the surface of stainless steel was evaluated. A dispersion of the construct was prepared at a concentration of 1 mg/mL in sterile deionised water. (It is noted that attempts to disperse the construct in saline resulted in precipitation of the construct.) A volume of 100 μL of the dispersion was dispensed onto the surface of a 1×1 cm stainless steel (SS 304) square. A control was prepared by dispensing the same volume of sterile deionised water onto the surface of a second stainless steel square. Both samples (test and control) were then dried at 60° C. for a period of two hours. The samples were stored at room temperature prior to use. A volume of 1 mL of an actively growing (log phase) culture of Escherichia coli (ATCC 25922) in 21 g/L Mueller-Hinton broth (MHB) was serially diluted (10⁻⁶) to provide 8 to 10 colony forming units (CFUs) per 100 μL. Individual samples of the stainless steel squares were placed in each well of a sterile 12-well culture plate and 100 mL of the serially diluted culture dispensed onto the surface of each sample. The culture was allowed to contact the surface for a period of 20 minutes at room temperature before washing each sample once with phosphate buffered saline (PBS) to remove non-adherent cells of the bacterium. Each washed sample was then immersed in a volume of 10 mL of MHB and incubated overnight at 37° C. Following overnight incubation each sample was washed as before and immersed in a volume of 9 mL of MHB. Alternate vortexing and sonicating was employed to remove bacteria from the sample surface. A volume of a serial dilution (10⁻⁴) of the resulting broth was then spread on blood agar plates, incubated at 37° C. overnight and colonies counted. Cell densities of the overnight cultures were calculated and are presented in Table 1.

TABLE 1
Growth of overnight cultures of Escherichia coli (ATCC 25922)
following contact with surface treated (Test) and untreated (Control)
of 1 × 1 cm stainless steel samples.

| Sample | CFU/mL |
|---|---|
| Control | No growth |
| Test | 3 × 10⁷ |

The tabulated results indicate a biocidal action of the samples treated with the polycation-lipid construct designated Spm-Ad-DOPE (9a).

The ability of the following constructs to prevent the growth of clinical isolates of Staphylococcus aureus and Staphylococcus epidermidis on the surface of stainless steel (SS 316) was evaluated:
The construct designated NCSeCH₂CO—CMG(2)-Ad-DOPE
The construct designated Spm-Ad-DOPE (9a)
The construct designated HA-gar-Ad-DOPE as described in the specification accompanying international application no. PCT/NZ2006/000245 [publ. no. WO 2007/035116].

Frozen stocks of the Staphylococcus sp. inocula were thawed at 37° C. for 10 minutes and vortexed before plating on blood agar and incubating at 37° C. for 18 hours. Single colonies were used to inoculate a volume of 10 mL of Mueller-Hinton Broth (MHB) and incubated at 37° C. for 18 hours in a shaking incubator (200 rpm). A volume of 100 μL of the actively growing culture was then used to inoculate a volume of 100 mL of MHB and incubated at 37° C. for 6½ hours. The turbid culture suspension (OD₆₀₀=1.596) was serially diluted to 10⁻⁸ in eight volumes of 9 mL of MHB. A volume of 100 μL of each dilution step was spread on plate count agar (PCA) plates and incubated at 37° C. for 18 hours to confirm viable cell numbers. The dilution to 10⁻⁷ was used in the following steps.

Volumes of 100 μL of dispersions in water of each construct were dispensed onto the surface of stainless steel squares (5 replicates);
2 mg/mL NCSeCH₂CO—CMG(2)-Ad-DOPE;
1 mg/mL Spm-Ad-DOPE (9a); and
1 mg/mL HA-gar-Ad-DOPE.

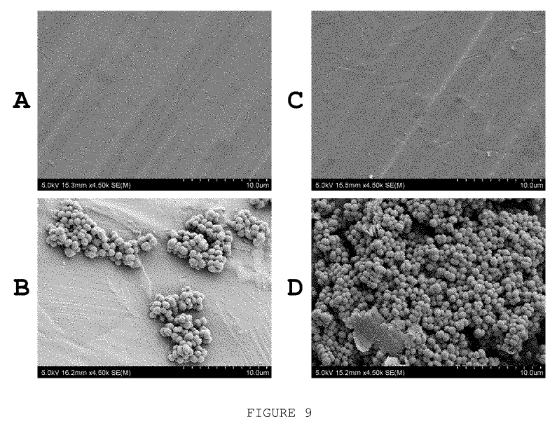

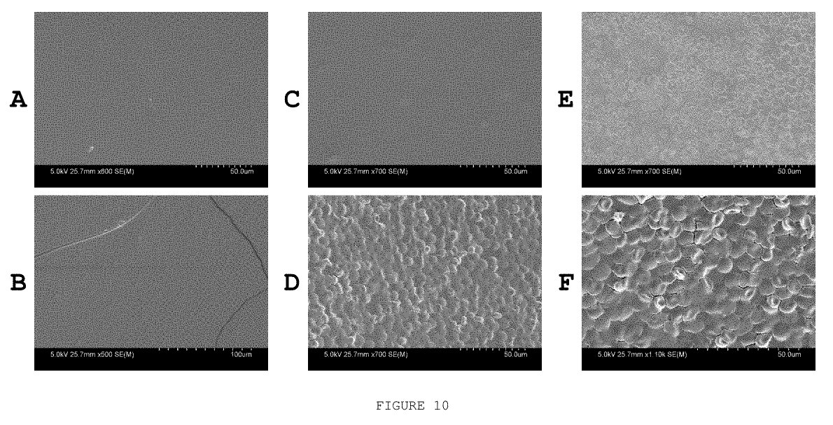

The stainless-steel squares were then dried at 60° C. for 30 minutes and kept at room temperature before use. Each one of the treated stainless steel squares was then placed in an individual well of a sterile multi-well plate and volumes of 100 μL of the dilution to 10⁻⁷ of actively growing Staphylococcus sp. isolate dispensed into each well. The plate was then incubated at 37° C. for 18 hours with shaking (200 rpm). The stainless steel squares were each then removed from their respective wells and washed 3 times in volumes of 1 mL of sterile Maximum Recovery Diluent (MRD) and then placed in a volume of 9 mL of MRD and serially diluted to 10⁻⁴ as before. For each dilution step a volume of 100 μL was spread on a PCA plate, allowed to dry and then incubated at 37° C. for 18 hours and colonies counted. The surface of the stainless steel squares was also examined by SEM following fixing in 2.5% glutaraldehyde (4° C. overnight), dehydration with increasing concentrations of ethanol, drying and sputter-coating with platinum for 60 seconds and imaging at 5.0 kV at 4,500× magnification. Ten fields selected at random were counted for each sample. Electron micrographs of randomly selected fields are provided in FIG. 8.

The susceptibility of clinical isolates of Staphylococcus aureus, Escherichia coli and Pseudomonas aeruginosa to the constructs designated Spm-Ad-DOPE (9a) and NCSeCH₂CO—CMG(2)-Ad-DOPE was determined by broth microdilution. Actively growing cultures of each clinical isolate were prepared as described above to obtain cultures with the following turbidities (OD₆₀₀):

| | |
|---|---|
| Staphylococcus aureus | 1.371 |
| Escherichia coli | 1.300 |
| Pseudomonas aeruginosa | 0.906 |

Serial dilutions of each culture to 10⁻⁶ were prepared and used as inocula. Volumes of 50 μL of each dilution was spread on PCA plates and incubated at 37° C. for 18 hours. A two-fold dilution series for each of the two constructs, spermine (2) and selenous acid (H₂SeO₃) were prepared in sterile 96-well plates. Volumes of 50 μL of inocula were dispensed into a well containing a volume of 50 μL of the serially diluted construct, spermine (2) or selenous acid (H₂SeO₃). Following gentle mixing the plates were incubated in the dark at 37° C. for 18 hours (200 rpm). Minimum inhibitory concentrations (MIC) were thereby observed and minimum bactericidal concentrations (MBC) determined by plating aliquots from each well. Both constructs were observed to be more effective bacteriostatic (Table 2) or bactericidal (Table 3) agents then either of their functional moieties (spermine or selenous acid) alone.

TABLE 2
Minimum inhibitory concentrations (MICs) as determined by
broth microdilution.

| | MIC reading in μM | | | |
|---|---|---|---|---|
| | | | Spm-Ad- | |
| | NCSeCH2CO- | | DOPE | Spermine |
| Clinical isolate | CMG(2)-Ad-DOPE | H₂SeO₃ | (9a) | (2) |
| S. aureus | 992.51 | 1985.01 | 236.63 | 473.26 |
| E. coli | 1985.01 | 1985.01 | 118.32 | 118.32 |
| P. aeruginosa | 1985.01 | >1985.01 | 236.63 | 3786.09 |

TABLE 3
Minimum bactericidal concentrations (MBCs) as determined by
broth microdilution and plating.

| | MBC reading in μM | | | |
|---|---|---|---|---|
| | | | Spm-Ad- | |
| | NCSeCH2CO- | | DOPE | Spermine |
| Clinical isolate | CMG(2)-Ad-DOPE | H₂SeO₃ | (9a) | (2) |
| S. aureus | 1985.01 | >1985.01 | 236.63 | 946.52 |
| E. coli | >1985.01 | >1985.01 | 118.32 | 118.32 |
| P. aeruginosa | >1985.01 | >1985.01 | 236.63 | >3786.09 |

Surface Treatment—Micro-Dimensioned Particle (Biotic Origin) Adherence

A stock solution of the construct designated Spm-Ad-DOPE (9a) was prepared in methanol at a concentration of 10 mg/mL. The stock solution was diluted to a concentration of 250 μg/mL in methanol and 25 μL volumes of the diluted stock solutions dispensed into each of the round bottomed wells of a multi-well microplate (Corning Inc.). The plates were allowed to dry before washing the wells 6 times with deionized water. Control wells were similarly treated using either methanol alone (blank) or substituting the construct designated Biotin-CMG(2)-Ad-DOPE as described in the specification accompanying international application no. PCT/NZ2008/000266 [publ. no. WO 2009/048343]).

Red blood cells (RBCs; group O, up to 2 weeks old) were washed and re-suspended at a concentration of 1% packed cell volume (pcv) in phosphate-buffered saline (PBS). A 50 μL volume of the suspension of RBCs was dispensed into each of the wells and incubated for 1 hour at room temperature before washing 6 times with PBS. The RBCs were fixed by adding a 50 μL volume of a solution of glutaraldehyde in PBS at a concentration of 2.5% (w/v) and incubating for 10 minutes before washing each well with water and allowing to dry. The RBCs were lysed by adding a 50 μL volume of deionized water and incubating for 10 minutes before discarding the water and allowing to dry.

For scanning electron microscopy (SEM) the bottom of each well was cut from the plate and the treated surface sputter coated with platinum prior to imaging. The images obtained at increasing magnification for well surfaces treated according to the methods described and those obtained for commercially available plates (Capture-R™ Ready-Id, Capture-R™ Ready-Screen and CT-6; Immucor Inc.) are provided in FIG. 10.

Adherence of RBCs to well surfaces treated according to the method described was clearly evident. Investigations were performed to determine if the adherence could be attributable to the use of the construct designated Spm-Ad-DOPE (9a) or the polycation spermine (2) alone. A stock solution of spermine (2) was prepared at a concentration of 10 mg/mL in methanol. The spermine stock solution was diluted to a concentration of 0.2 mg/mL in either methanol or water. Volumes of 200 μL of diluted stock solution (approximately 950 μM) of the construct designated Spm-Ad-DOPE (9a), spermine (2) in methanol or spermine in water were added to each of the first wells of a microplate. A two-fold serial dilution from each of the first wells was then performed. The microplate was then dried under vacuum before washing each well of the microplate with deionized water by immersing and discarding the wash water 4 times. After drying, 50 μL volumes of a suspension of RBCs at a concentration of 1% pcv in either 10 mM Tris/0.25 M sucrose (SucT) or PBS were dispensed into each well. (Aggregation of RBCs was observed in wells where the construct designated Spm-Ad-DOPE (9a) had been added at a concentration of 29 μM or greater.) The plate was incubated for one hour at room temperature before washing the wells 6 times with PBS.

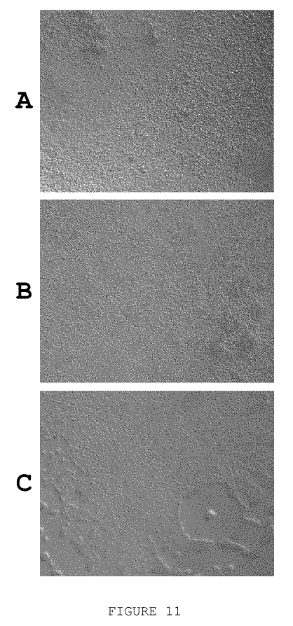

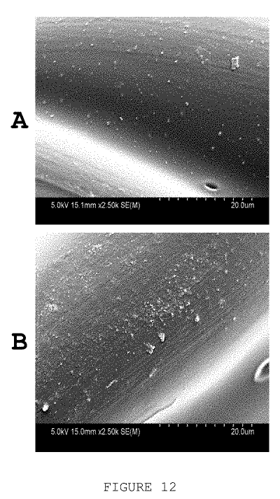

Following drying the plate was inverted and the base of the wells examined by light microscopy (100× magnification) for the presence of a uniform monolayer of RBCs. Photomicrographs obtained for wells treated with a solution of either the construct designated Spm-Ad-DOPE (9a) or spermine (2) alone are presented in FIG. 11. A uniform monolayer of cells was observed in wells treated with the construct designated Spm-Ad-DOPE (9a) at concentrations of 14 to 237 μM (0.015 to 0.25 mg/mL) in either of the two buffers (SucT or PBS) used. Any attachment of cells when spermine (2) alone was used was not uniform or reproducible. No cells were observed to adhere to the surface in wells containing spermine (2) alone in water.

Surface Treatment—Micro-Dimensioned Particle (Abiotic Origin) Adherence

A volume of 100 μL of a solution of 0.05% (w/v) bromophenol blue and 50 μM of the construct designated KODE-spm in water was dispensed and spread across the surface of a strip of laminated nylon mesh. The strip was allowed to dry for one hour at room temperature before being exposed to particulates released from either smoking cigarettes or a wood burner using an artificial syringe as a “puffer” (exposure for about 10 minutes). The exposed strips were stored in a sealed polythene bag before being examined by scanning electron microscopy (SEM) (FIG. 12).

Although the invention has been described with reference to embodiments or examples it should be appreciated that variations and modifications may be made to these embodiments or examples without departing from the scope of the invention. Where known equivalents exist to specific elements, features or integers, such equivalents are incorporated as if specifically referred to in this specification. In particular, variations and modifications to the embodiments or examples that include elements, features or integers disclosed in and selected from the referenced publications are within the scope of the invention unless specifically disclaimed. The advantages provided by the invention and discussed in the description may be provided in the alternative or in combination in these different embodiments of the invention.

### INCORPORATION BY REFERENCE

The application accompanied by this specification claims the benefit of the priority dates established by the filing of Australian application nos. 2014904423 (filed 3 Nov. 2014) and 2015901844 (filed 20 May 2015) and international application nos. PCT/NZ2015/050181 (filed 3 Nov. 2015) and PCT/IB2016/052735 (filed 12 May 2016). The descriptions provided in the specifications accompanying each of these applications, and those of the specifications accompanying international application nos. PCT/NZ2006/000245 [publ. no. WO 2007/035116] and PCT/NZ2008/000266 [publ. no WO 2009/048343], are incorporated here.

### PUBLICATIONS

Behr et al (1989) Efficient gene transfer into mammalian primary endocrine cells with lipopolyamine-coated DNA Proc. Natl. Acad. Sci. USA, 86, 6982-6986

Blagbrough et al (1997) Polyamines and polyamine amides as potent selective receptor probes, novel therapeutic lead compounds and synthetic vectors in gene therapy Pharmaceutical Sciences, 3, 223-233

Bovin et al (2008) Functional Lipid Constructs international PCT application no. PCT/NZ2008/000266 (publ. no. WO 2009/048343 A1)

Byk et al (1998) Synthesis, activity, and structure-activity relationship studies of novel cationic lipids for DNA transfer J. Med. Chem. 1998, 41, 224-235

Clauss (1970) Stabilized bath for deposition of copper by chemical reduction U.S. Pat. No. 3,492,135

Distler (1967) The chemistry of Bunte salts Angew. Chem. Internat. Edit. Vol. 6, No. 6, 544

Gallo et al (2014) Antibacterial Surface Treatment for Orthopaedic Implants Int. J. Mol. Sci. 2014, 15, 13849-13880

Geall and Blagbrough (2000) Homologation of polyamines in the rapid synthesis of lipospermine conjugates and related lipoplexes Tetrahedron 56, 2249-2460

Gunn (2007) Organotellurium and Selenium-Based Antimicrobial Antimicrobial [sic] Formulations and Articles international PCT application no. PCT/US2007/064333 (publ. no. WO 2007/109633 A2)

Gunn (2008) Organotellurium and Selenium-Based Antimicrobial Formulations and Articles U.S. patent application Ser. No. 11/688,230 (publ. no. US 2008/0031931 A1)

Jeney and Zsolnai (1959) Bacteriostatic action of organic selenocyanates Naturwissenschaften, 46, 231

Kato et al (2003) Immobilized culture of nonadherent cells on an oleyl poly(ethylene glycol) ether-modified surface BioTechniques 35, 1014-1021

Kerstetter and Gramlich (2014) Nanometer-scale self-assembly of amphiphilic copolymers to control and prevent biofouling J. Mater. Chem. B, 2014, 2, 8043-5052

Kruszewski et al (2013) Reducing Staphylococcus aureus biofilm formation on stainless steel 316L using functionalized self-assembled monolayers NIH Public Access Author Manuscript Mater Sci Eng C Mater Biol Appl 33(4): 2059-2069

Numao et al (1981) Showdomycin analogues: Synthesis and antitumor evaluation J. Med. Chem. 1981, 24, 515-520

Randazzo et al (2009) A series of cationic sterol lipids with gene transfer and bactericidal activity Bioorganic & Medicinal Chemistry 17, 3257-3265.

Reid and Spallholz (2007) Selenium-Based Biocidal Formulations and Methods of Use Thereof U.S. patent application Ser. No. 11/439,751 (publ. no. US 2007/0224275 A1)

Reid and Spallholz (2007) Selenium-Based Biocidal Formulations and Methods of Use Thereof international PCT application no. PCT/US2006/020310 (publ. no. WO 2007/008293 A2)

Reid and Spallholz (2010) Selenium-Based Biocidal Formulations and Methods of Use Thereof U.S. patent application Ser. No. 12/669,440 (publ. no. US 2010/0158966 A1)

Reid et al (2009) Anti-Microbial Orthodontic Compositions and Appliances and Methods of Production and Use Thereof U.S. patent application Ser. No. 12/460,046 (publ. no. US 2010/0028823 A1)

Reid et al (2009) Anti-Microbial Orthodontic Compositions and Appliances and Methods of Production and Use Thereof International PCT application no. PCT/US2009/004053 (publ. no. WO 2010/080086 A1)

Reid et al (2010) Selenium-Based Biocidal Formulations and Methods of Use Thereof U.S. patent application Ser. No. 12/669,460 (publ. no. US 2010/0158967 A1)

Reid et al (2012) Anti-Microbial Orthodontic Compositions and Appliances and Methods of Production and Use Thereof U.S. patent application Ser. No. 13/556,282 (publ. no. US 2012/0288813 A1)

Reid et al (2012) Anti-Microbial Orthodontic Compositions and Appliances and Methods of Production and Use Thereof U.S. Pat. No. 8,236,337

Reid et al. (2013) Selenium-Based Biocidal Formulations and Methods of Use Thereof U.S. patent application Ser. No. 13/762,147 (publ. no. US 2013/0165595 A1)

Remy et al (1994) Gene transfer with a series of lipophilic DNA-binding molecules Bioconjugate Chem. 5, 647-654

Shi et al (2012) Antibacterial and osteoinductive capability on orthopedic materials via cation-π interaction mediated positive charge Journal of Materials Chemistry B, 2014, 00, 1-5

Zsolnai (1962) Discovery of new fungicides. IV. Organic sulfur compounds Biochemical Pharmacology, 11, 271-297

1. An antimicrobial surface treatment method comprising the step of contacting the surface of an object with an aqueous dispersion of at least one functional-lipid construct where the lipid is a di-acyl, di-alkenyl or di-alkyl glycerophospholipid and the functional moiety of the construct is selenide.

2. The method of claim 1 where the object is a surgical dressing or implant.

3. The method of claim 2 where the object is a surgical implant.

4. The method of claim 3 where the surface is stainless steel.

5. The method of claim 1 where the aqueous dispersion is devoid of detergents and organic solvents.

6. The method of claim 5 where the aqueous dispersion consists of saline or water and the at least one functional-lipid construct.

7. The method of claim 1 where the lipid is a di-acyl glycerophospholipid.

8. The method of claim 7 where the lipid is a phosphatidylethanolamine.

9. The method of claim 8 where the lipid is a di-oleoyl phosphatidyl-ethanolamine.

10. The method of claim 1 where the functional moiety is cyanoselenide.

11. The method of claim 1 where the antimicrobial surface treatment is an antibacterial surface treatment.

12. The method of claim 11 where the antimicrobial surface treatment is a bactericidal surface treatment.

13. The method of claim 1 where the contacting the surface is by immersing the object in the dispersion for a time sufficient to provide the antimicrobial surface treatment.

14. The method of claim 13 where the time is less than 60 seconds.

15. The method of claim 14 where the time is less than 30 seconds.

16. The method of claim 15 where the time is less than 10 seconds.

17. The method of claim 16 where the dispersion is sonicated whilst the object is immersed.

18. A selenide-lipid construct of the structure: 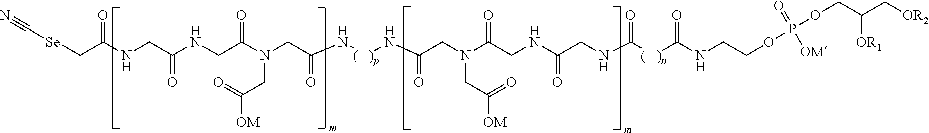
where:
m is the integer 1, 2, 3 or 4;
n is the integer 3, 4 or 5;
q is the integer 1, 2 or 3;
M is a monovalent substituent;
M′ is a monovalent cation or substituent; and
R₁ and R₂ are independently an aliphatic C₁₄₋₂₀ acyl, aliphatic C₁₄₋₂₀ alkenyl or aliphatic C₁₄₋₂₀ alkyl substituent.

19. The construct of claim 18 where m is the integer 1, 2 or 4.

20. The construct of claim 19 where m is the integer 2, n is the integer 4, p is the integer 2, M is the monovalent substituent H, M′ is the monovalent cation H⁺, and R₁ and R₂ are the aliphatic C₁₈ alkenyl substituent oleoyl.
